(19) United States
(12) Patent Application Publication
(10) Pub. No.: US 2007/0010819 A1
(43) Pub. Date: Jan. 11, 2007

(54) Bone fixing device and method for distracting a fracture and jig and method for insertion of a bone fixing device

(75) Inventor: Alan John Johnstone, Aberdeen (GB)

Correspondence Address: Gregory J. Lavorgna;Drinker Biddle & Reath, One Logan Square, 18th & Cherry Streets, Philadelphia, PA, 19103-7595, US

(73) Assignees: GRAMPIAN HEALTH BOARD, Aberdeen (GB); ROBERT GORDON UNIVERSITY, Aberdeen (GB)

(21) Appl. No.: 10/570,224

(22) PCT Filed: Aug. 31, 2004
(86) PCT No.: PCT/GB04/03742

§ 371 Date: Feb. 28, 2006

(30) Foreign Application Priority Data
Aug. 30, 2003 (GB) 0320375.9

(51) Int. Cl. A61B 17/58

(52) U.S. Cl. 606/73

(57) ABSTRACT

A bone fixing device and a method of distracting a fracture are described. The bone fixing device has threads at both a leading end and a trailing end, and the pitch of the threads are greater at the trailing end. This causes the leading end of the bone fixing device to travel more slowly through the bone than the trailing end, which loads the threads of the screw to put the screw under compression, so that the threads bite more effectively into the bone. A jig and method for insertion of two bone fixing devices are also described. The jig defines parallel planes of insertion which cross when viewed in a direction perpendicular to the parallel planes.

The present invention concerns an apparatus and method for treating a fracture in a bone, especially but not exclusively for treating a wrist fracture.

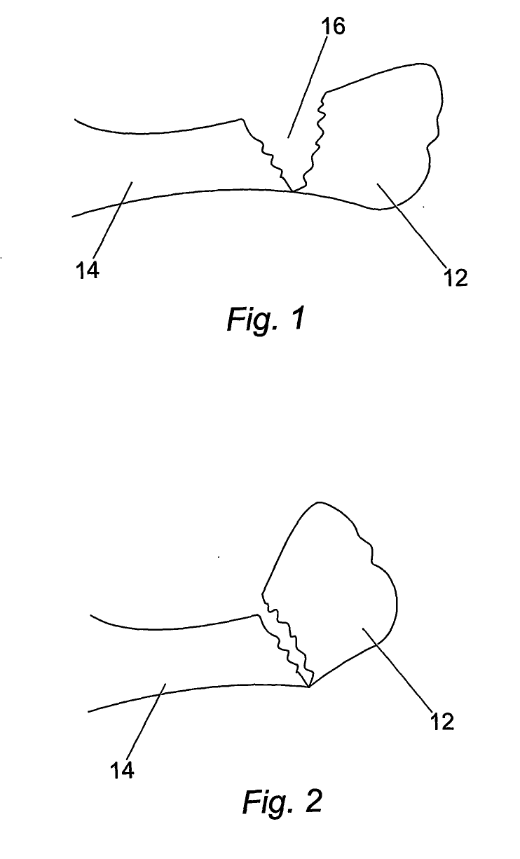

When a wrist bone breaks, it often fractures into many fragments on the dorsal side of the wrist, whilst leaving a fairly clean break on the lower side. A typical broken wrist is shown in FIG. 1. Distal bone portion 12 needs to be held fixed relative to proximal bone portion 14 in the position shown in FIG. 1, so that the bone portions 12, 14 do not heal in the position shown in FIG. 2. Further bone fragments generally float in the space designated 16. These will fuse with bone portions 12, 14 as the fracture heals.

One method of treating a wrist fracture involves attaching a metal frame to the outside of the wrist, spanning the wrist joint. Pins typically extend from the frame into the bone portions on each side of the fracture. This can be an invasive method, and can immobilise the joint for a long period, resulting in a very stiff joint once the fracture has healed. A further alternative method involves fixing the fracture using plates and screws. All of these methods are very invasive, in some cases totally preventing movement of the joint and are likely to be very inconvenient to the patient.

According to the present invention there is provided a bone fixing device for distracting a fracture, the bone fixing device having a body with a leading end and a trailing end, the bone fixing device being threaded at least at the leading end and at the trailing end; wherein the pitch of the threads at the trailing end is greater than the pitch of the threads at the leading end.

By “distracting a fracture” we mean causing a partial movement of fracture fragments away from one another.

Not allowing the bone portions to move towards one another is advantageous, especially for fractures such as that shown in FIG. 1, where the lower side of the fracture is a clean break, and the other side is broken into multiple fragments. If the bone portions were not maintained by the fixing device, the bone portions could pivot around the clean break and out of alignment.

Distraction of the fracture (i.e. loading the bone portions to push the bone portions apart relative to each other) has been discovered to be desirable, as putting the bone portions in tension causes the bone portions to grip the threads of the fixing device more firmly, which reduces the likelihood of the fixing device working itself loose. Distracting the fracture loads the threads of the screw so that the screw is under compression and the threads bite more effectively into the bone.

The greater the pitch of the screw threads, the quicker the axial progress through the bone. Therefore, if the pitch of the screw threads at the trailing end (e.g. at the head) of the fixing device is greater than the pitch at the leading end (e.g. at the tip that is driven through the bone first), the trailing end will progress more quickly through the bone than the leading end, which will push the two bone portions apart to distract the fracture.

In the following description, the “leading” end and the “tip” end both refer to the end of the fixing device which penetrates the bone first. The “trailing” end and the “head” end both refer to the opposite end of the fixing device from the leading end.

Typically, the fixing device is self-drilling and has a self-tapping tip.

Optionally, the trailing end of the fixing device does not have a radially-extending head, i.e. unlike a typical screw, the head of the fixing device is simply an axial extension/continuation of the leading end of the fixing device; which is flush with the rest of the fixing device.

Optionally, the trailing end of the fixing device has a larger diameter than the leading end.

Typically, the fixing device diameter increases uniformly towards the trailing end. Alternatively, the fixing device comprises two portions of different diameters.

Typically, threads are provided along the entire length of the fixing device. Alternatively, threads are provided only at the leading and trailing ends of the fixing device.

Optionally, the screw threads increase uniformly in pitch towards the head of the fixing device. Optionally, the fixing device comprises two portions each having screw threads of a different pitch.

Optionally, the screw threads have a longitudinal axis and at least one side of at least one of the screw threads has a portion steeply inclined relative to the axis.

“Steeply inclined” is intended to encompass both a side which is perpendicular to the axis and a side having a portion that is steeply sloping. “Steeply inclined” can include the meaning substantially perpendicular.

The screw threads having one or more steeply inclined portions make the fixing device difficult to remove from the bone on application of an axial force, without rotating the fixing device. This is especially advantageous for use in soft bone, (such as in elderly patients) where the fixing device could otherwise accidentally fall out or fail to hold the fracture reduction.

Typically, the screw threads include at least one side steeply inclined relative to the axis in each axially-facing direction. Typically, each thread has a steeply inclined side in at least one axially-facing direction. Typically, the steeply inclined sides face the opposing ends of the fixing device. Typically, the other sides of the threads slope more gradually than the steeply inclined sides, and these sloped sides typically face one another i.e. towards the centre of the fixing device.

Typically, the screw is made from a bioabsorbable material. The bioabsorbable material may comprise polylactic acids, polyglycolic acids, or a combination of both. Alternatively, the screw is made from metal. The invention is not limited to any of these exemplary materials.

Preferably, the screw has a coating, e.g. a dip coating. Typically, the coating comprises hydroxyapatite. The presence of hydroxyapatite (naturally found in bone tissue) encourages new bone growth in the surrounding vicinity of the screw.

According to a second aspect of the present invention there is provided a method of treating a fracture by distracting the fracture to hold the bone portions apart under tension.

The method is optionally achieved by inserting a bone fixing device according to the first aspect of the invention into the bone.

Optionally, the fixing device is cannulated and the method includes the steps of inserting a guide wire into the bone and subsequently inserting the fixing device over the guide wire.

Optionally, the fixing device is self-drilling and/or has a self-tapping tip and the bore is formed by the fixing device, rather than a drill.

Optionally, the method includes using a jig to aid alignment of the fixing device and/or any guide wire and/or any drill or tap used.

According to a third aspect of the invention there is provided a fixing device for insertion into at least two bone portions to treat a fracture, the fixing device having a body with formations on its outer surface adapted to maintain the relative position of the bone portions; and an axial bore extending through the body.

According to a fourth aspect of the invention there is provided a fixing device for insertion into at least two bone portions to treat a fracture, the fixing device having a body with formations on its outer surface adapted to maintain the relative position of the bone portions; and a self-tapping tip.

According to a fifth aspect of the invention there is provided a method of treating a fracture, comprising engaging a fixing device into at least two bone portions of the fracture to maintain the relative position of the bone portions.

According to a sixth aspect of the present invention there is provided a fixing device for insertion into at least two bone portions to treat a fracture, the fixing device having a body with formations on its outer surface adapted to distract the bone portions.

According to a seventh aspect of the present invention there is provided a method of treating a fracture, comprising inserting a fixing device into at least two bone portions of the fracture, whereby insertion of the fixing device holds the two bone portions apart under tension to distract the fracture.

This invention also relates to a jig to aid insertion of a bone fixing device into a bone to treat a fracture. This invention relates especially but not exclusively to a jig which aids the relative alignment of at least two bone screws.

Bones often fracture into a plurality of bone fragments. In this case, it is helpful to insert two screws along different line of insertion, so as to span most of or all of the fragments. It is highly undesirable if these two screws collide with each other, because this could prevent the second screw from being fully inserted into the bone, and could also force the first screw out of position. If this happens, the second screw would have to be removed and a new hole made.

Therefore, to avoid this problem, the second screw is typically inserted at a safe distance away from the first screw so that there is a negligible chance of a collision. However, once the screws have been inserted, the only support keeping the screw in the bone is friction between the bone and the screw threads. In patients with fragile crumbly bone, e.g. some elderly patients, this friction may not be sufficient, and the bone screws may work themselves loose. This could further damage the bone and additional surgery may be required.

According to a eighth aspect of the invention there is provided a jig for positioning first and second bone fixing devices, the jig defining first and second lines of insertion for the first and second bone fixing devices respectively, wherein the lines of insertion lie in parallel planes and wherein the lines of insertion cross when viewed in a direction perpendicular to the parallel planes.

Provision of the jig allows the second line of insertion to be defined very accurately with respect to the first line of insertion. Because the lines of insertion are in parallel planes, they can never intersect, and the possibility of bone fixing devices colliding with each other is reduced.

Optionally, more than two lines of insertion could be defined by the jig.

Typically, the bone fixing devices are at least partially threaded. Preferably, the bone fixing devices comprises bone screws.

Preferably, the parallel planes are relatively close to one another. Preferably, the parallel planes have a minimum separation equal to the diameter of the bone fixing device. If the fixing device comprises a threaded fixing device, e.g. a bone screw, preferably the parallel planes have a minimum separation equal to the outer thread diameter of the bone fixing device. Such embodiments can ensure that the bone fixing devices will not collide.

Some such embodiments can provide that the bone fixing devices interact, thus providing an additional connection to each other, which helps to hold the bone fixing devices in the bone. “Interact” is used in the sense that the bone fixing devices can touch and can lean against each other, one fixing device effectively buttressing and supporting the other.

Preferably, the orientation of the second line of insertion is variable relative to the orientation of the first line of insertion. Preferably, the second line of insertion is translatable relative to the first line of insertion.

“Translation” is used throughout the specification to mean not only movement along a straight line, but also movement along an arcuate path. “Translational movement” is used as a way of differentiating between rotational movement about an axis.

Preferably, the jig includes an arm that extends in a lateral direction with respect to at least one of the lines of insertion.

Preferably, the arm is arcuate.

Preferably, at least a part of the arm has a planar surface that is parallel to the parallel planes of the lines of insertion. optionally, at least a part of the arm has a planar surface that is co-planar with one of the parallel planes of the lines of insertion.

Preferably, the first and second lines of insertion are defined by respective first and second guide means coupled to the arm. Typically, the first and second guide means have bores that serve to align the path of the guide wire or bone fixing device with the desired line of insertion. In some embodiments, the guide means may include at least one guide sleeve received in the bore of the guide means and the guide sleeve can be slidably and/or rotatably mounted in the bore so that it is releasably coupled to the arm. Typically, the arm is rotatably mounted relative to the guide means so that the arm can pivot around the axis of the guide means.

A guide means can be anything which can guide the path of a guide wire and/or a bone fixing device. Typically, the first and second guide means can include first and second guide sleeves. Optionally, each of the first and second guide means comprises a plurality of concentric sleeves. However, in more basic embodiments, an apertured section of the arm can suffice, and separate guide sleeves are not necessary.

Typically, the first and second guide means are adapted to receive guide wires and/or bone fixing devices. The invention is not limited to the use of guide wires because in some embodiments, the first and second guide means can directly guide the bone fixing devices into the bone.

Optionally, the jig includes a spacer adapted to space the parallel planes of the lines of insertion apart from each other.

Typically, the spacer is a separate component which is coupled to the arm.

Optionally, the spacer comprises a block having a guide bore adapted to receive a guide means. Preferably, the guide means can be slidably mounted in the guide bore, and can optionally rotate relative thereto.

Optionally, the spacer has an attachment bore and the spacer is coupled to the arm by an attachment device inserted through the attachment bore in the spacer and also into/through the arm. Preferably, the spacer is rotatably mounted on the arm. Typically, the attachment device comprises a bolt and a nut; the bolt and nut connection can be loosened to allow rotation of the spacer relative to the arm. After selection of the required angle, the nut can be tightened to fix the rotational position of the spacer relative to the arm.

Optionally, an elongate aperture is provided in the arm, and the spacer is moveable along the elongate aperture. If the attachment device is a bolt and a nut, the bolt can be loosened to allow translation of the spacer relative to the arm using the elongate aperture, and then tightened again to restrict further translational movement once a position has been selected.

Optionally, the arm may be extendible to allow relative translational movement of the first and second guide means.

Typically, the position of one of the guide means is defined by the arm and the position of the other guide means is defined by the spacer, such that rotating the spacer relative to the arm varies the orientation of the second guide means relative to the first guide means, and translating the spacer along the arm varies the distance between the first and second guide means.

Rotational and translational capabilities are not essential. Embodiments where the first and second lines of insertion are fixed relative to each other are also covered by the invention (for example, if the first and second guide means are both fixed relative to the arm).

The spacer is not necessarily a separate component. Alternatively, the spacer could comprise a part of the arm. For example, the arm could comprise a first portion and a second portion having parallel planar surfaces, wherein the second portion is stepped relative to the first portion. In such embodiments, the step in the arm can function as a spacer to space the two parallel surfaces of the first and second parts of the arm from each other, and these parallel surfaces can define the parallel planes of the first and second lines of insertion.

In such embodiments, the position of the second guide means may be fixed in angular orientation and/or translational position relative to the first guide means (apart from any optional slidable mounting to the arm). For example, such an embodiment may be made by attaching a first guide means to one end of the arm and a second guide means to the other end of the arm on the other side of the stepped portion. However, in preferred embodiments, at least one of the guide means is rotatably mounted on the arm. This could be achieved, for example, by hingedly mounting the second guide means to the arm. In this way, the relative angles of insertion of the two bone fixing devices can be selected.

In some embodiments, a stepped arm and a separate spacer component can both be used in conjunction to provide the two parallel planes of the lines of insertion.

In all embodiments, the arm may optionally have an elongate slot to enable relative translational movement of the first and second guide means.

In all embodiments, the arm may optionally be extendible to enable relative translational movement of the first and second guide means.

According to a ninth aspect of the present invention there is provided a method of positioning first and second bone fixing devices relative to each other, the method including the steps of defining first and second lines of insertion for the first and second bone fixing devices respectively; wherein a jig is used to define the position of the second line of insertion relative to the first line of insertion such that the lines of insertion lie in parallel planes and such that the lines of insertion cross when viewed in a direction perpendicular to the parallel planes.

Preferably, this method is performed using the jig of the eighth aspect of the invention.

Preferably, the separation of the parallel planes is greater than or equal to the diameter of the bone fixing devices. Optionally, the fixing devices are at least partially threaded and the separation of the parallel planes is greater than or equal to the outer thread diameter of the bone fixing devices.

Optionally, the bone fixing devices are inserted at locations very close to each other. Optionally, the bone fixing devices are inserted at locations in which they are in contact with each other. Optionally, the bone fixing devices are at least partially threaded, and the threads are in contact with each other or narrowly miss each other.

Typically, the jig includes first and second guide means which define respective lines of insertion of the first and second bone fixing devices, and the method includes the step of translating and/or varying the orientation of one guide means relative to the other. Preferably, the method includes the step of fixing the two guide means at a selected orientational and translational position. The guide means can be guide sleeves.

Optionally one or each of the first and second guide means comprises a plurality of concentric sleeves. An inner sleeve may be used to insert a guide wire. An intermediate sleeve may be used to insert a drill over the guide wire to drill a hole for the fixing device. An outer sleeve may be used to guide the insertion of the fixing devices (and any optional tap used). Alternatively, the fixing devices may be self drilling/self tapping, rendering the use of a drill/tap and the intermediate guide sleeve unnecessary.

As apparent from the above, the jig does not include the guide sleeves, as the guide sleeves are themselves attachable to and removable from the jig. Any guide sleeves/drill sleeves can be used with the jig.

Typically, the method includes the step of inserting first and second guide wires into the bone along respective first and second lines of insertion. Typically, the position of the first line of insertion is selected and the first guide wire and then optionally the first fixing device are inserted without using the jig (e.g. using standard drill/guide sleeves). The jig is then fitted over the projecting part of the guide wire and used to define the second line of insertion for the second guide wire and the second fixing device, which are then inserted using the second guide means.

Typically the method includes the additional step of inserting first and second bone fixing devices over the first and second guide wires into the bone.

Typically, at least the second fixing device is inserted using the jig, for example by inserting the second fixing device through a guide sleeve coupled to the jig. The guide sleeve protects the surrounding tissues from damage by the fixing device and the drill. Alternatively, the jig may be removed before insertion of one or both of the fixing devices, and a separate guide sleeve(s) may be used to guide the fixing device along the lines of insertion already defined by the guide wires.

Alternatively, the first and second bone fixing devices could be inserted via the guide means into the bone, without the need for any guide wires.

According to a tenth aspect of the present invention there is provided a method of positioning first and second bone fixing devices in a bone such that they just touch each other.

Embodiments of the invention will now be described, by way of example only, and with reference to the following drawings, in which:

FIG. 1 shows a side view of a broken wrist with bone portions in the correct alignment to heal;

FIG. 2 shows a side view of a broken wrist, with bone portions incorrectly aligned;

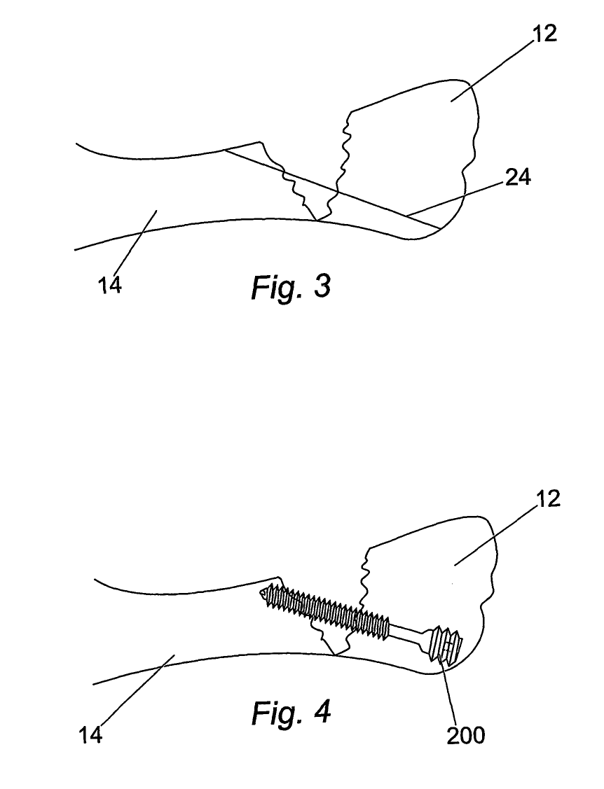

FIG. 3 shows a cross-section of two bone portions with a guide wire inserted through both bone portions;

FIG. 4 shows a cross-section of the bone portions of FIG. 3, with a fixing device inserted through the portions;

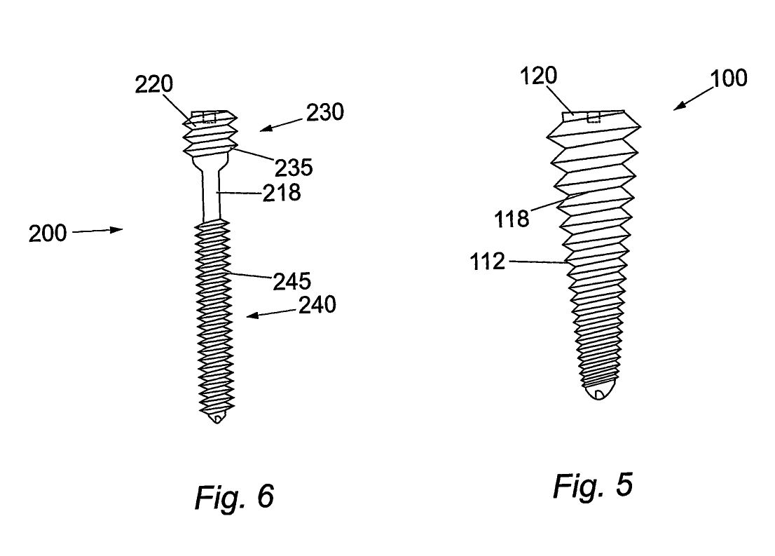

FIG. 5 shows a side view of a further alternative embodiment of a fixing device having threads of varying pitch;

FIG. 6 shows a side view of a further alternative embodiment, having threads of different pitches and two portions of different diameters;

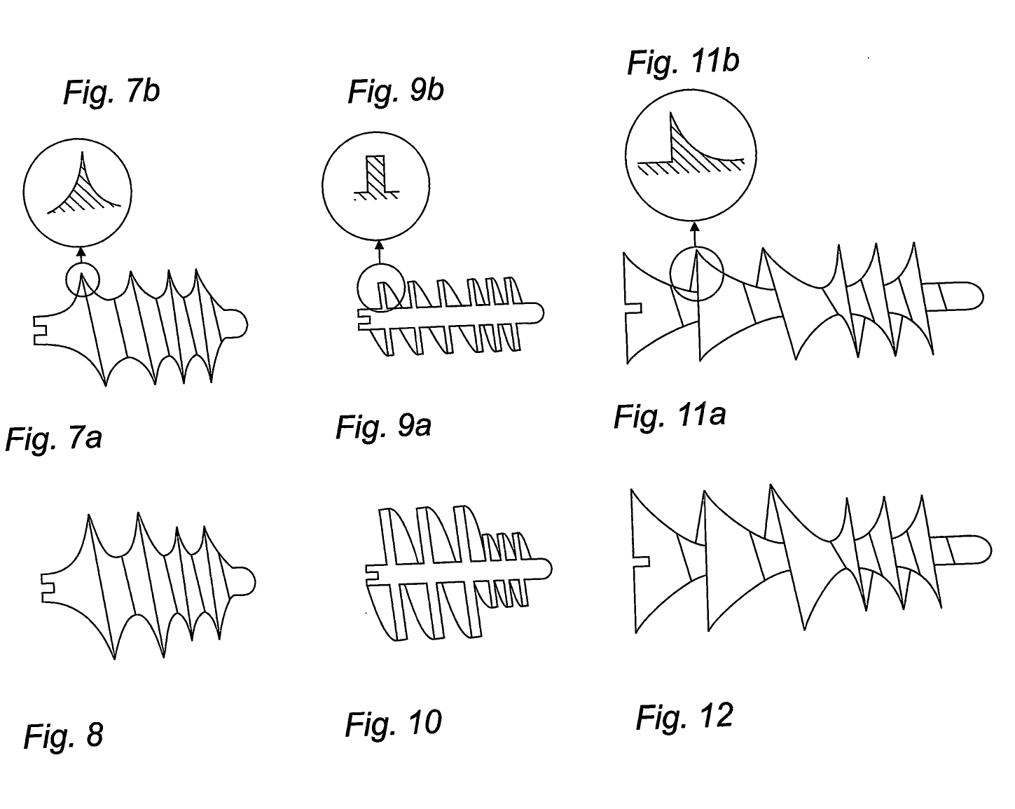

FIGS. 7a and 7b show a further embodiment, having steeply sloping screw threads of varying pitch;

FIG. 8 shows a side view of a further embodiment, having steeply sloping screw threads of varying pitch and a varying diameter;

FIGS. 9a and 9b show sectional views of a further embodiment, having rectangular threads of varying pitch;

FIG. 10 shows a sectional view of a further embodiment, having rectangular threads of varying pitch and varying diameter;

FIGS. 11a and 11b show a further embodiment, having perpendicular screw threads in each axially-facing direction, the threads having a varying pitch;

FIG. 12 shows a side view of a further embodiment, having perpendicular screw threads in each axially-facing direction of varying pitch and varying outer diameter;

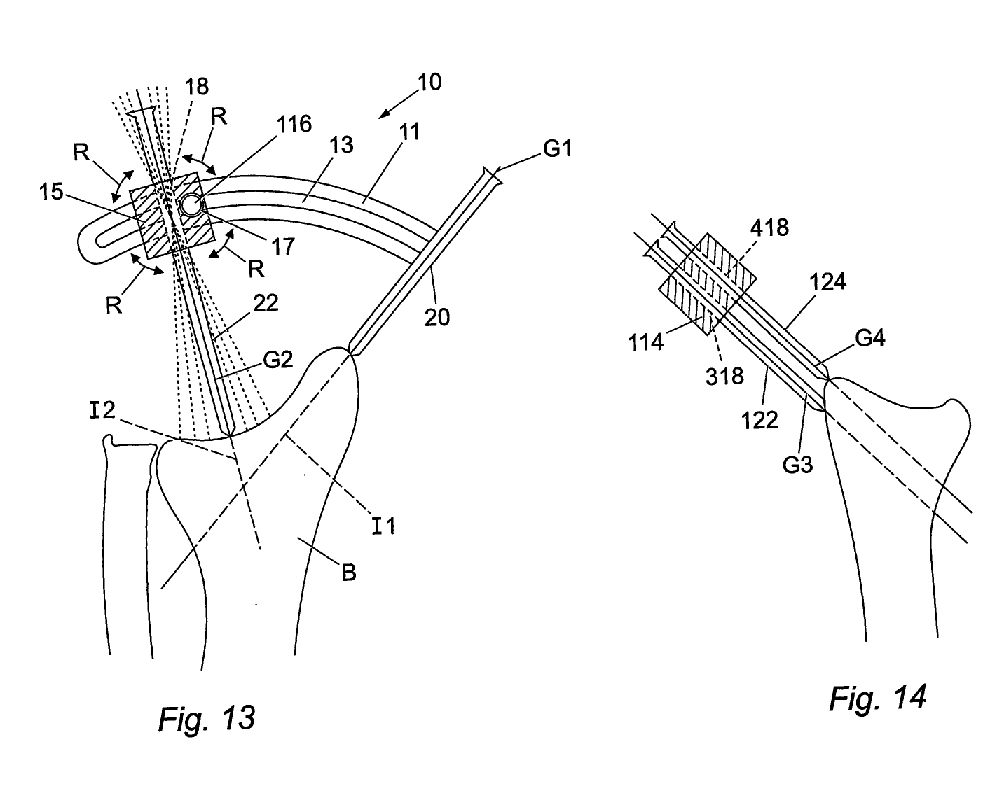

FIG. 13 shows a cross-sectional view of a jig of the present invention being used to insert guide wires into a bone;

FIG. 14 shows a cross-section view of part of a second embodiment of the invention;

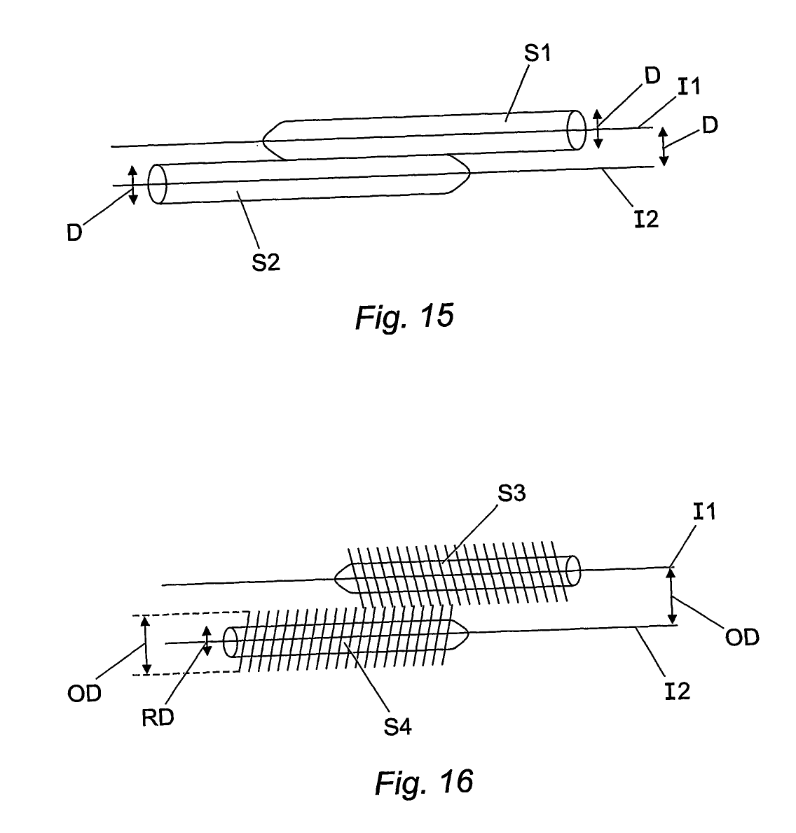

FIG. 15 shows a schematic view from above of two fixing devices inserted along respective lines of insertion I1, I2, wherein the distance apart of the parallel planes of the lines of insertion equals the diameter of the fixing device;

FIG. 16 shows a schematic view similar to FIG. 15, wherein the fixing devices are threaded and the spacing of the parallel planes equals the outer (thread) diameter of the fixing devices;

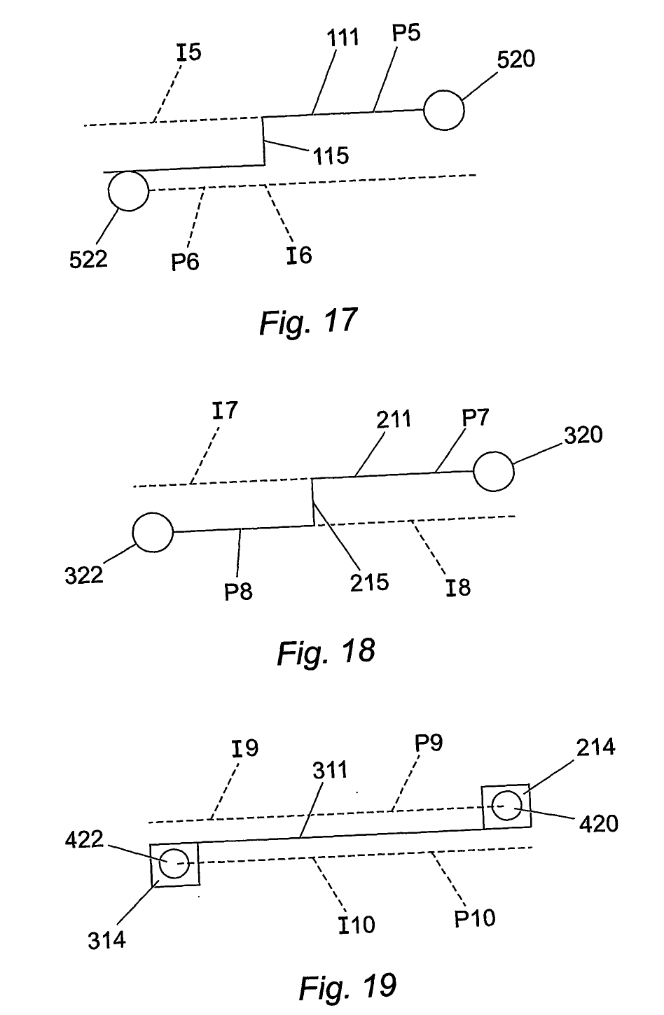

FIG. 17 shows a schematic top view of a modified jig having a stepped arm;

FIG. 18 shows a schematic top view of a modification to the FIG. 17 stepped arm jig;

FIG. 19 shows a schematic top view of an alternative jig having two spacers and a non-stepped arm;

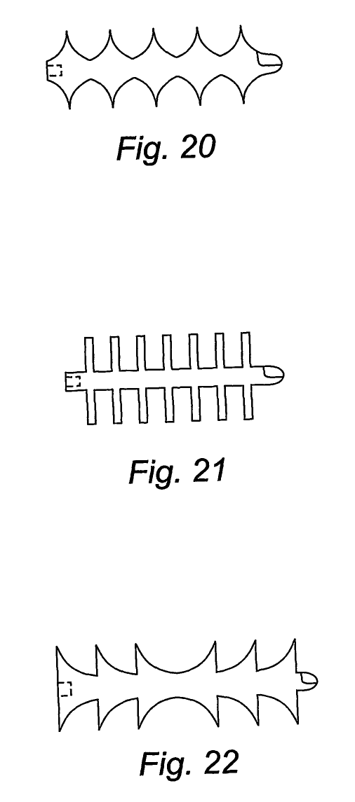

FIG. 20 shows a sectional view of a further embodiment, having steeply sloping screw threads of consistent pitch;

FIG. 21 shows a sectional view of a further embodiment, having rectangular threads of consistent pitch; and

FIG. 22 shows a sectional view of a further embodiment, having perpendicular screw threads in each axially-facing direction.

Referring now to the drawings, FIG. 5 shows a screw 100 having a head 120, a root 118 and screw threads 112 all the way along its length. The screw 100 is tapered, i.e. the diameter of root 118 and the outer diameter of screw threads 112 typically both increase uniformly towards the head 120. Such tapering can allow the screw 100 to be more easily inserted into a pilot hole; in some cases, a pilot hole may not even be required. Furthermore, the increasing diameter of the root 118 and the threads 112 towards the head end of screw 100 will urge the root 118 against previously uncut portions of bone, and the threads will cut into new bone which was not previously cut by the smaller diameter threads, both of which will secure the screw 100 more firmly into the bone than could be achieved with other designs.

The pitch of screw threads 112 increases uniformly towards the head 120. The pitch of the threads is typically in the range 1 mm to 2 mm at the leading (tip) end, and 2 mm to 4 mm at the trailing (head) end. The screw 100 will typically cause a relative distraction of bone portions 12, 14 of 1 mm to 2 mm per rotation.

The head 120 is flush with the outer surface of root 118 so that the head 120 is an axial continuation of the body of the screw 100 and it does not project radially outward of the root 118. Alternative embodiments may have an outwardly projecting head. In this example, screw 100 is cannulated to allow insertion over a guide wire (not shown). However, non-cannulated embodiments can also be used. The head 120 may have an aperture for insertion of an Allen key or other torque tool. The screw 100 preferably has a self-drilling, self-tapping tip.

Although screw 100 shown here has screw threads 112 extending along the whole of its length, alternative embodiments (not shown) may have screw threads only at particular portions, and not at others, for example, at leading and trailing ends, with a mid-portion remaining unthreaded.

Alternatively, the root diameter could be constant, and the outer thread diameter alone could increase towards the head 40 (examples of such embodiments are found in FIGS. 8, 10 and 12, described below).

FIG. 6 shows a screw 200 (having a root 218 and a head 220), which is similar to screw 100, in that the root 218 and threads vary in diameter. However, instead of varying uniformly, screw 200 is divided into two portions of different diameter: a trailing portion 230 having screw threads 235 and a leading portion 240 having screw threads 245. Both the diameter of the root 218 and the outer diameter of the screw threads 235, 245 are different between each of the two portions, but are uniform within each portion 230, 240. The trailing portion 230 has a larger diameter root (of around 2 mm to 4 mm) and a larger outer diameter of screw thread (of around 3 mm to 6 mm) than the leading portion 240 (3 mm to 5 mm and 4 mm to 7 mm for root and thread respectively). Threads 230 have a constant pitch, as do threads 240, but the pitch of screw threads 230 is approximately 2-4 mm, which is greater than the pitch of screw threads 240 at approximately 1-2 mm. Thus, the diameter of the root and the threads, and the thread pitch varies in the same way as for screw 100, except that the variation is abrupt between the two portions 230, 240, instead of continuous. As with screw 100, embodiments of screw 200 may be cannulated; may have a self-drilling, self-tapping tip; may have a non-threaded mid-portion; and may have a head which does not project outwardly of its root. The arrangement shown in FIG. 6 could of course be modified so that the sides of the root of the screw 200 are parallel so that the diameter of the root is uniform for the length of the screw 200. Such a modification could optionally have the variations in the diameter and pitch of the threads that are shown in FIG. 6.

In use, the bone portions 12, 14 are first aligned into the correct position shown in FIG. 1. Next, a K-wire 24 is typically inserted into the bone to span the fracture, with opposite ends of the K-wire 24 engaging respective bone portions 12, 14. An image intensifier (apparatus which produces low intensity x-rays) is typically used to check that the K-wire 24 is correctly positioned, as shown in FIG. 3. The length of protruding guide wire is measured and deducted from the known length of the wire to indicate the correct screw length.

Next, the screw 100, 200 is inserted over the K-wire 24 and screwed into the bone portions 12, 14, e.g. by using an Allen key, as shown in FIG. 4. If the screw 100, 200 is not self-drilling and/or self-tapping, then a hole would have to be drilled and/or tapped before inserting the screw 100, 200 e.g. with a cannulated drill inserted over K-wire 24. A jig is optionally used to align the screw 100, 200 and/or the K-wire, and/or any drill used.

The leading end (tip end) of the screw 100, 200 engages bone portion 14 and the trailing end (head end) engages bone portion 12. Since the head 120, 220 is merely an extension of the body of screw 100, 200 and it does not protrude radially outward from the root 118, the head 120, 220 can be driven completely inside bone portion 12 along with the rest of screw 100, 200 (see FIG. 4), as opposed to complete insertion being prevented by a radially-extending head abutting against the exterior surface of cortical bone portion 12.

If a screw having a radially-extending head were to be fully inserted into bone portion 12, when the head abuts the exterior of bone portion 12, the head 120, 220 would be held stationary with respect to bone portion 12, whilst the leading end of screw 100, 200 would travel further into bone portion 14. This would pull the bone portions 12, 14 together, compressing the fracture, as opposed to maintaining or distracting it.

It would be possible to have a radially-extending head and to avoid the above problem of compression, by using an alternative embodiment (not shown) wherein the screw 100, 200 is long enough for the head not to abut the outer surface of bone portion 12 when the rest of the screw is correctly positioned in bone portions 12 and 14; this embodiment would typically not be fully inserted into the bone portions.

The greater the pitch of the screw threads, the greater the distance the screw will advance into the bone per rotation of the screw. Therefore, if a screw has screw threads of different pitches on different parts of the screw, these different parts of the screw will travel at different rates. The increase in pitch of the screw threads 112, 230, 240 towards the head 120, 220 means that for every rotation of the screw 100, 200, the tip of the screw will progress more slowly through the bone than the head 120, 220. Therefore, the leading end of the screw 100, 200, with its low-pitch screw threads will advance more slowly through bone portion 14, compared to the trailing (head) end of the screw 100, 200 advancing through bone portion 12. This will result in the two bone portions 12, 14 being pushed apart, and the screw 100, 200 being put into compression between the two bone portions. The more the screw is rotated, the more the bone portions 12, 14 are pushed apart to distract the fracture.

For example, in the case of screw 200, if the pitch of the screw threads 245 is twice the pitch of the screw threads 235, the screw threads 245 will advance into the bone twice as quickly as screw threads 235, so for every 1 mm of axial movement of the leading portion 230, there will be 2 mms of axial movement of the trailing portion 240 and therefore the bone portions 12, 14 will have become pushed apart (distracted) by 1 mm.

The screw 100, 200 is driven through the bone until the bone portions have been distracted sufficiently. The screw 100, 200 can now be left in the bone in that position until the fracture has healed, or permanently as clinically indicated.

Maintaining the position of the bone portions is advantageous, especially for fractures such as that shown in FIG. 1, where the lower side of the fracture is a clean break, and the other side is broken into multiple fragments. If the relative positions of the main bone portions are not maintained by the fixing device, and they are instead pulled together, the bone portions could pivot around the clean break, displace the comminuted fragments in the space 16, and heal out of alignment.

Distraction of the fracture (i.e. loading the bone portions to push the bone portions apart relative to each other) has been discovered to be desirable, as putting the bone portions in tension causes the bone portions to grip the formations (e.g. screw threads) of the fixing device more firmly, which reduces the likelihood of the fixing device working itself loose. Distracting the fracture loads the threads of the screw so that the screw is under compression and the threads bite more effectively into the bone.

Referring now to FIGS. 7 to 12, there are shown screws having modified thread profiles. FIG. 7b shows an enlarged cross-sectional view of a single screw thread of FIGS. 7a and 8. Similarly, FIG. 9b shows an enlarged cross-sectional view of a single screw thread of FIGS. 9a and 10. FIG. 11b shows an enlarged cross-sectional view of a single screw thread of FIGS. 11a and 12.

In the screws of FIGS. 7 and 8, both the leading and trailing sides of the thread slope between the radially outer tip of the thread and the root; the slope is very steep. The steeper the slope (i.e. the closer to perpendicular with the root axis) the more difficult it is to remove the screw from the bone by an axial force. This is because the near-perpendicular surfaces provide an abrupt resistance to the passage of the screw through the bone, as compared to more gradually sloping surfaces, where the screw would use the gradual slope to ease its way through the bone. Therefore the screws of FIGS. 7 and 8 would be very difficult to remove from the bone, in either axial direction.

The FIG. 7 screw threads vary in pitch, with the pitch increasing towards the trailing end (head end). The pitch could vary uniformly, or abruptly. Thus, the FIG. 7 screw is suitable for distracting a fracture.

The FIG. 8 screw has two portions of different root and outer thread diameter, wherein the thread pitch on the leading portion is smaller than that of the trailing portion. The pitch of the threads is typically in the range 1 mm to 2 mm at the leading (tip) end, and 2 mm to 4 mm at the trailing (head) end. The FIG. 8 screw will typically cause a relative distraction of bone portions 12, 14 of 1 mm to 2 mm per rotation.

The screws of FIGS. 9 and 10 exhibit an alternative design of screw thread, wherein the threads have sides that are substantially perpendicular to the body. The substantially perpendicular sides are shown particularly well in FIG. 9b. Like the design of threads of FIGS. 7 and 8, a screw with these threads would be very difficult to remove from a bone by axial force in either direction.

The FIG. 9 screw has a constant root diameter, a constant outer thread diameter, but a varying pitch of thread. The FIG. 9 screw comprises two discrete portions of different pitch of thread; however, alternative embodiments could have a uniformly varying pitch. The pitch of the threads is typically in the range 1 mm to 2 mm at the leading (tip) end, and 2 mm to 4 mm at the trailing (head) end. The FIG. 9 screw will typically cause a relative distraction of bone portions 12, 14 of 1 mm to 2 mm per rotation.

The FIG. 10 screw has a constant root diameter, a varying thread pitch, and a varying outer diameter of the thread. The FIG. 10 screw comprises two discrete portions of different thread pitch and outer thread diameter; however, alternative embodiments could have a uniformly varying pitch/outer diameter. Alternative embodiments also having a varying root diameter could also be used. The pitch of the FIG. 10 threads is typically in the range 1 mm to 2 mm at the leading (tip) end, and 2 mm to 4 mm at the trailing (head) end. The FIG. 10 screw will typically cause a relative distraction of bone portions 12, 14 of 1 mm to 2 mm per rotation.

The screws of FIGS. 11 and 12 show a further alternative design of screw thread. Each thread has one perpendicular side and one sloping side; the threads at the leading end of the screw have perpendicular leading sides, and the threads at the trailing end of the screw have perpendicular trailing sides. The perpendicular sides are shown particularly well in FIG. 11b.

The FIG. 11 screw has threads of varying pitch. The leading and trailing ends of the FIG. 11 screw have threads of a constant outer diameter and a constant root diameter. The FIG. 11 screw comprises two discrete portions of different thread pitch; however, alternative embodiments could have a uniformly varying pitch. Alternative embodiments also having a varying root diameter could also be used.

The FIG. 12 screw has a constant root diameter, a varying thread pitch, and a varying outer diameter of the thread. The FIG. 12 screw comprises two discrete portions of different thread pitch and outer thread diameter; however, alternative embodiments could have a uniformly varying pitch/outer diameter. Alternative embodiments also having a varying root diameter could also be used. The pitch of the FIG. 12 threads is typically in the range 1 mm to 2 mm at the leading (tip) end, and 2 mm to 4 mm at the trailing (head) end. The FIG. 12 screw will typically cause a relative distraction of bone portions 12, 14 of 1 mm to 2 mm per rotation.

As explained above, the steeper the angle of the thread, the greater the resistance of the screw to removal in the direction of that edge. Steeply sloping thread edges with respect to the root (like those of the FIGS. 9 and 10 embodiments) will provide an abrupt resistance to axial movement, as compared to more smoothly sloping edges, which would ease the axial movement of the screw using the principal of the inclined plane. Therefore, the threads at the leading end will provide a high resistance to movement in the direction of insertion, due to the perpendicular leading edges. The threads at the trailing end will provide a high resistance to axial movement in the direction opposite to the direction of insertion, due to their perpendicular trailing edges. Thus, the screws of FIGS. 11 and 12 resist removal in both axial directions, due to the leading end threads resisting axial movement in the direction of insertion, and the trailing end threads resisting axial movement in the opposite direction.

The screws of FIGS. 7 and 12 all provide a high resistance to axial force once in the bone, due to having at least some steep edges relative to the axis of the screw in both axial directions. This is particularly useful for use in fragile bones, for example in the elderly, where the bone is so soft that a conventional screw could more easily be pulled through the bone.

All the screws of FIGS. 7 to 12 are suitable for distracting a fracture, as they all have screw threads of a smaller pitch at the leading end as compared to the trailing end.

It should be noted that it is not only the FIG. 5 or FIG. 6 design (varying root diameter) that has the advantage that the trailing threads cut into previously uncut bone. The embodiments having a constant root diameter but increased outer diameter of the threads at the trailing end (such as those shown in FIGS. 8, 10 and 12) also have this advantage. The larger diameter threads at the trailing end cut into previously uncut bone to secure the screw more firmly in the bone.

The bone fixing devices of all embodiments may be metallic or non-metallic, and the invention is not limited to the use of any particular material. The bone fixing device may be made of a bioabsorbable material. Examples of suitable bioabsorbable materials include polylactic acids and polyglycolic acids; however any other medically recognised bioabsorbable material could be used.

The fixing device may or may not be provided with a coating. A possible example of a suitable coating is hydroxyapatite. This can be applied to the fixing device by dipping the fixing device in the coating and allowing the coating to dry. Hydroxyapatite is a common calcium salt that is also found naturally in bone. The presence of a hydroxyapatite coating on the fixing device encourages the surrounding bone to grow, both on to the fixing device itself and, generally, in the vicinity of the screw.

In some useful versions of the fixing device, the threads need not vary between the ends of the device, and FIGS. 20-22 show embodiments that maintain the loading on the screw by means of the shapes of the threads, rather than by means of the changes in pitch. Despite the lack of change in pitch of the thread which can be used for active loading of the threads in a distracted fracture, the embodiments of FIGS. 20-22 will be subjected to loading by the natural bias of the bones tending to compress the device, which will thereby exert a loading force on the threads of the screw tending to resist dislodgement of the screw when implanted. Referring now to FIG. 20, there is shown a screw having modified thread profiles similar to that shown in FIG. 7, although the FIG. 20 screw has a constant root diameter, a constant pitch thread and a constant outer thread diameter.

In the FIG. 20 screw, both the leading and trailing sides of the thread slope between the radially outer tip of the thread and the root; the slope is very steep, and is typically non-linear, in that the slope is more gradual near the root of the screw, and steeper nearer to the radial edge of the thread. The steeper the slope (i.e. the closer to perpendicular with the root axis) the more difficult it is to remove the screw from the bone by an axial force. This is because the near-perpendicular surfaces provide an abrupt resistance to the passage of the screw through the bone, as compared to more gradually sloping surfaces, where the screw would use the gradual slope to ease its way through the bone. Therefore the FIG. 20 screw would be very difficult to remove from the bone, in either axial direction.

FIG. 21 is another version of a screw with rectangular threads similar to the FIG. 9 embodiment, but having a constant root diameter, a constant pitch thread and a constant outer thread diameter. This screw is suitable for maintaining a fracture.

FIG. 22 shows a further version of screw with a thread profile similar to that of the FIG. 11 embodiment, but having a constant root diameter, a constant pitch thread and a constant outer thread diameter. This screw is suitable for maintaining a fracture.

Modifications and improvements may be incorporated without departing from the scope of the invention. For example, any of the screws shown in FIGS. 7 to 12 could optionally have a non-threaded mid-portion (not shown), and/or a varying root diameter (not shown).

Screw 200 and the screws of FIGS. 8, 10 and 12 all have an abrupt change of diameter; however, the diameter change could alternatively be uniform, such as in screw 100. Any of the designs of screw threads shown in FIGS. 7 to 12 could be combined with any of screws 100 and 200.

All of the screws in FIGS. 7 to 12 can have self-drilling, self-tapping tips; they may be cannulated; they may have non-threaded mid-portions; and they may have a head which does not project outwardly of the surface of the root.

Although the examples here are described with reference to wrist fractures, the invention could be used for any fracture.

FIG. 13 shows a jig 10 which is being used to define two lines of insertion of two threaded bone fixing devices (not shown) into a bone B. In this example, bone B is a radius, and the fixing devices are to be inserted in the head of the radius. However, the invention is not limited to the use in any particular part of any particular bone.

The head of bone B is typically fractured (fracture lines not shown), and the fixing devices (e.g. bone screws) are to be inserted to hold the fracture fragments together.

The jig 10 has an elongate arm 11 which has the form of an arc. The arm 11 has a slot 13 which extends along the length of the arm 11. The slot 13 extends all the way through the arm 11.

A first sleeve 20 is coupled to one of the elongate ends of the arm 11 such that the arm 11 extends from a lateral side of the sleeve 20. The first sleeve 20 may be coupled to the arm 11 by any suitable means. Preferably, the sleeve 20 is slidably mounted on the arm, for example the arm 11 may have a guide slot and the sleeve 20 may be slidably mounted in the slot. The sleeve 20 has an inner bore which is adapted to receive a first guide wire G1. The first sleeve 20 may be a standard guide/drill sleeve, but any guide means (typically with an aperture to receive the guide wire or bone fixing device) can be used.

A spacer 15 in the form of an alignment block 15 is coupled to the arm 11 by a bolt 116. The spacer 15 is cuboid, and has a bore 17 in which the bolt 116 is received. The bolt 116 also passes through the slot 13. A nut (not shown) engages one end of the bolt 116 to hold the spacer 15 fixed relative to the arm 11.

When the nut is loosened, the bolt 116 can move within the slot 13 so that the spacer 15 moves relative to the slot 13; the spacer 15 can also be rotated around the axis of the bolt 116 in the plane defined by the arcuate arm 11. This rotation is shown in FIG. 13 by the arrows R.

The spacer 15 also has a further bore 18, which extends through the spacer 15 in a direction perpendicular to the bore 17. The bore 18 goes directly between opposite sides of the cuboid spacer 15 in a straight line which is perpendicular to the opposite sides (not at an inclined angle through the block). Therefore, the bore 18 lies in a plane parallel to the plane of the elongate arm 11. The bore 18 is adapted to receive a second sleeve 22. Preferably, the second sleeve 22 is slidably mountable and optionally rotatably mountable in the bore 18. The second sleeve 22 is similar to the first sleeve 20, and has an inner bore adapted to receive a second guide wire G2.

Although just single sleeves 20, 22 are shown in FIG. 13, each of these can represent a set of concentric sleeves. For example, sleeve 22 can represent an inner sleeve for a guide wire, an intermediate sleeve for a drill and an outer sleeve for a fixing device. The same applies to sleeve 20.

The second sleeve 22 defines a second line of insertion I2 and the first sleeve 20 defines a first line of insertion I1. The lines of insertion I1, I2 are continuations of the axes of the sleeves 20, 22 into the bone.

The lines of insertion I1, I2 lie in parallel planes, because their relative positions are defined by the sleeves 20, 22, which are in turn defined by the jig 10. The first line of insertion I1 always lies in the plane of the elongate arm 11, as the first sleeve 20 is fixed (apart from any slidable mounting) to the end of the arm 11, the arm 11 extending from a lateral side of the first sleeve 20.

As explained above, the bore 18 lies in a plane parallel to the plane of the elongate arm 11; therefore the second line of insertion I2 always lies in a plane parallel to the plane of the elongate arm 11. This is always true independent of the selected rotational and translational position of the spacer 15. In the FIG. 13 view, the plane of the second line of insertion I2 is in front of the plane of the elongate arm 11.

The second line of insertion I2 is adjustable relative to the first line of insertion I1 by altering the rotational and translational position of the spacer 15. The angles of the guide sleeves 20, 22 can therefore be adjusted so that the lines of insertion I1, I2 cross when viewed in a direction perpendicular to the parallel planes of the lines of insertion I1, I2 (as seen in the FIG. 13 view).

Because the arm 11 is arcuate (see FIG. 13) no additional rotational adjustment of the spacer 15 relative to the slot 13 is necessary in order to ensure that the lines of insertion cross. For example, in some embodiments, rotation of the spacer is guided by the arcuate slot 13 so that on moving (translating) the spacer 15 along the slot 13, the spacer 15 is automatically rotated so that the axis of the bore 18 is always perpendicular to the part of the slot 13 in the vicinity of the spacer 15. This could be achieved by providing the spacer 15 with one or more lateral projections adapted to engage the slot 13. These embodiments can provide jigs which are simple to operate, since a change in translational position of the spacer along the arcuate jig automatically adjusts the spacer's rotational position so that the respective lines of insertion cross in the desired location when seen from the FIG. 13 direction. However, the capability to rotate the spacer 15 is advantageous, as this allows a very precise selection of the angle of insertion (several alternative options for the second line of insertion are shown in dotted lines in FIG. 13).

The position of the bore 18 in the spacer 15 is preferably selected such that the distance between the plane of the elongate arm 11 (the plane of the first line of insertion) and the parallel plane in which the bore 18 lies (the plane of the second line of insertion) is greater than or equal to the diameter of the bone fixing device (the outer thread diameter, if threaded). The reason for this will be explained with reference to FIGS. 15 and 16. In FIG. 15, D represents the diameter of the fixing devices, and in FIG. 16, RD represents the root diameter of the fixing devices, and OD represents the outer (thread) diameter of the fixing devices.

FIG. 15 shows a view from above of two non-threaded bone fixing devices S1, S2 which have been inserted along respective lines of insertion I1, I2. The tips of the fixing devices and the lines of insertion I1, I2 are angled into the page. The lines I1, I2 also represent the positions of the parallel planes of the lines of insertion in this view. In FIG. 15, the distance between the parallel planes is equal to the diameter D; therefore the fixing devices will just touch, but they will not collide.

FIG. 16 shows a further embodiment where the bone fixing devices S3, S4 are threaded. In this embodiment, the distance between the parallel planes is equal to the outer (thread) diameter OD, and the outer threads of the inserted fixing devices will just touch but not interlock.

Both FIGS. 15 and 16 show how two bone fixing devices can be inserted into positions where they just touch but do not collide.

This is advantageous, as one fixing device is able to lean against the other; one fixing device effectively buttressing the other. This provides additional support for the bone fixing devices, which helps to keep them in the bone. In the threaded embodiments this support is additional to the friction between the threads and the bone. The fixing devices do not have to be literally touching each other to achieve this effect; bone fixing devices a small distance apart could also achieve this effect. The invention encompasses both literally touching embodiments and other embodiments in which the bone fixing devices pass close together.

FIG. 15 illustrates that the bone fixing devices are not necessarily threaded, and can be simple rods.

In use, the position of the first line of insertion is selected and a first guide wire G1 is inserted (e.g. using a guide sleeve) to define the first line of insertion I1. Next, a hole is drilled for the first fixing device using a cannulated drill (and also typically a drill sleeve). Next, a first fixing device is inserted into the bone over the guide wire G1 into the hole drilled by the drill (optionally using a tap if the fixing device is not self-tapping). In this method, typically an appropriate sized guide/drill sleeve is selected for each step.

Next, the guide sleeves 20, 22 are coupled to the jig 10 and this assembly is then positioned as shown in FIG. 13, with the first sleeve 20 being inserted over the protruding part of the guide wire G1. A screwdriver may be held in the first sleeve 20 so that it engages the first fixing device during the subsequent steps, to provide additional stability to the assembly whilst the second line of insertion is defined.

Next the nut (not shown) is loosened and the spacer is moved relative to the slot 13 to a desired translational and rotational position so that the second guide sleeve 22 defines a second line of insertion I2.

The surgeon typically ensures that the second line of insertion I2 crosses the first line of insertion I1 when viewed in a direction perpendicular to the parallel planes of the lines of insertion I1, I2 (i.e. in the direction of the front view of FIG. 13).

Once the second line of insertion I2 has been determined, the nut is tightened against the bolt to fix the rotational and translational position of the second sleeve 22. A second guide wire G2 is inserted through the second sleeve 22 and into the bone along the second line of insertion I2.

As explained above, the second sleeve 22 can represent a set of concentric sleeves, and the second guide wire G2 is typically inserted through an inner sleeve. Next, the inner sleeve can be removed, and a hole is drilled in the bone using a cannulated drill inserted over the second guide wire G2 using an intermediate-sized sleeve to guide the drill. Next, that sleeve is removed and an outer sleeve is used to guide the insertion of the second fixing device into the bone. If the fixing device is not self-tapping, a tap can be used to create space for a thread in the bone before insertion of the second fixing device; the tap can also be guided by the outer sleeve.

The bone fixing devices are typically cannulated bone screws S3, S4 and the guide wires G1, G2 guide the paths of the bone fixing devices in the bone. Because the lines of insertion have been accurately defined by the jig 10, the fixing devices S3, S4 can be positioned such that they pass closely together, without colliding (optionally even touching each other). This provides additional support to prevent the fixing devices S3, S4 coming loose and falling out of the bone. One fixing device effectively buttresses the other, which strengthens the connection of both fixing devices to the bone.

In a slightly modified method, the jig 10 and guide sleeves 20, 22 can be used to insert the first guide wire G1 and the first fixing device into the bone, instead of the jig 10 first being used after these have been inserted.

In some versions of the method, the jig 10 and sleeve 20 may be used to insert the first fixing device but not the first guide wire G1.

In all methods, it is advantageous to use some sort of guide sleeve (whether or not attached to the jig 10) to aid insertion of the guide wires, drill and fixing devices, to protect the surrounding tissues.

FIG. 14 shows an alternative embodiment of the invention, where the spacer 114 is modified to include more than one guide bore. In this embodiment, two parallel guide bores, 318, 418 are provided. The guide bores 318, 418 are adapted to receive respective sleeves 122, 124, which define lines of insertion of respective guide wires G3, G4. Because the guide bores 318, 418 are parallel, the lines of insertion they defined will not intersect and screws inserted through sleeves 122, 124 will not collide with each other. The guide bores 318, 418 may both lie in a single plane parallel to the plane of the arm 11 (i.e. side by side in the FIG. 13 view), or the guide bores 122, 124 may each lie in different planes parallel to the plane of the arm 11 (i.e. on in front of the other in the FIG. 13 view). The FIG. 14 embodiment may be particularly useful when the bone has fractured into many fragments, as including an extra fixing device can help to ensure that more of the fracture fragments are held together.

FIGS. 17 to 19 show alternative embodiments of the invention. FIG. 17 shows a modified arm 111 which includes a stepped portion 115. The stepped portion 115 divides the arm 111 into two portions which lie in parallel planes P5, P6; the stepped portion is an intermediate part of the arm 111 which is perpendicular to both portions of the arm 111. When seen from the front (like the FIG. 13 view), the P6 plane would be in front of the P5 plane. Therefore in this embodiment, the stepped arm 111 functions as a spacer, creating two parallel planes without the need for a separate spacer component 114.

First and second sleeves 520, 522 are attached to the arm 111. Only the top part of the sleeves 520, 522 are shown for clarity, but the sleeves actually extend downwards into the paper and towards each other, along the lines of insertion I5, I6. The first sleeve 520 is attached to the arm 111 so that the arm 111 extends from a lateral side of the sleeve 520 (similarly to the FIG. 13 embodiment). Therefore, the first sleeve defines a line of insertion I5 which lies in a plane P5 which is coplanar with the plane its end of the arm 111.

The second sleeve 522 is attached to a lateral side of the arm 111. The second sleeve 522 may be clamped onto the arm 111 by a clamp (not shown) that extends around the end of the arm 111. The second sleeve 522 may be rotatably and/or slidably mounted on the arm 111, or alternatively it may be fixed relative to the arm 111. Since the second sleeve 522 is attached to a lateral side of the arm 111, the line of insertion I6 defined by the sleeve 522 will lie in a plane P6 which is in front of the plane P6 of that end of the arm 111 (when viewed from the front). However, the line of insertion I6 is parallel to the plane P6. Therefore, the lines of insertion I5, I6 lie in respectively parallel planes.

FIG. 18 shows a further embodiment having an arm 211 with a stepped portion 215 defining two arm portions in parallel planes P7, P8, and first and second sleeves 320, 322 attached (e.g. slidably mounted) to the arm. This embodiment is the same as the FIG. 17 embodiment in most respects, except that the second sleeve 322 is attached to the arm 211 so that the arm 211 extends from a lateral side of the second sleeve 322. Therefore, in this embodiment, the lines of insertion I7, I8 defined by the sleeves 320, 322 will both lie along the parallel planes P7, P8 defined by the arm 211. The first and second sleeves 320, 322 are not rotatably mounted on the arm 111. However, in alternative embodiments, one or both of the first and second sleeves 320, 322 are rotatably mounted.

FIG. 19 shows a further embodiment of a jig having a non-stepped arm 311. The arm 311 is provided with two spacers 214, 314, which may be substantially the same as either of the spacers 15, 114 described above. The spacers 214, 314 are provided with respective sleeves 420, 422. In this view, the spacers 214, 314 are located at or near opposite ends of the arm, but one or both spacers is preferably translatable along the length of the arm 311 (e.g. by providing a slot as shown in the FIG. 13 embodiment).

The spacers 214, 314 are provided on opposite planar faces of the arm 311, and define two lines of insertion I9, I10, both of which lie in mutually parallel planes P9, P10; these planes are also parallel to the arm 311. At least one of the spacers 214, 314 is rotatable relative to the arm 311, so that the lines of insertion I9, I10 can be made to cross when viewed in a direction perpendicular to the parallel planes P9, P10. Thus, in this embodiment, the two spacers 214, 314 together define the spacing of the parallel planes.

In some embodiments, a stepped arm and a separate spacer component can both be used in conjunction to provide the two parallel planes of the lines of insertion.

The arms 111, 211, 311 could be either straight or arcuate when viewed from a front view perpendicular to the top view shown in these figures.

Modifications and improvements may be incorporated without departing from the scope of the invention. For example, the jig 10 need not be in the shape of an arc. In an alternative embodiment, the jig 10 could be straight.

In some embodiments, the first guide sleeve could be rotatably mounted on the arm and the second guide sleeve could be rotationally fixed with respect to the arm.

In some embodiments, the sleeves 20, 22 can comprise components of the jig 10, e.g. one or both of the sleeves may be permanently attached to the jig 10. In other embodiments, the sleeves 20, 22 are attachable to and removable from the jig 10 (e.g. by being slidably mounted on the arm/spacer). Although not shown in FIG. 13, an eye-member could be provided on the end of the arm 11, so that the first sleeve 20 can be slidably mounted in the eye member.

In further alternative embodiments, the arm could be extendible. An extendible arm could provide an alternative to a slotted arm, as this would also enable relative translation of the two guide sleeves. In some embodiments, the arm may be both slotted and extendible.

In further alternative embodiments, the jig is adapted to allow insertion of bone fixing devices at locations spaced apart by significant distances. Such embodiments provide an increased choice of relative angles and positions of the two fixing devices. In these embodiments, the fixing devices do not necessarily touch or even come close to each other, and the surgeon can still be confident that no collision will occur.

As illustrated in FIG. 15, the bone fixing devices are not necessarily threaded, and can be rods.

1. A bone fixing device for distracting a fracture, the bone fixing device having a body with a leading end and a trailing end, the bone fixing device being threaded at least at the leading end and at the trailing end;
wherein the pitch of the threads at the trailing end is greater than the pitch of the threads at the leading end.

2-3. (canceled)

4. A bone fixing device as claimed in claim 1, wherein the trailing end has a head which is flush with the rest of the body.

5. A bone fixing device as claimed in claim 1, wherein the body has a greater diameter at the trailing end than at the leading end.

6-9. (canceled)

10. A cannulated bone fixing device as claimed in claim 1.

11-12. (canceled)

13. A bone fixing device as claimed in claim 1, wherein the bone fixing device has a longitudinal axis and at least one side of at least one of the threads has a portion that is steeply inclined relative to the longitudinal axis.

14. (canceled)

15. A bone fixing device as claimed in claim 13, wherein each thread has a side having a steeply inclined portion facing the nearest end of the screw to that thread.

16-18. (canceled)

19. A bone fixing device as claimed in claim 13, including at least one thread having a side having a steeply inclined portion facing one axial direction and at least one thread having a side having a steeply inclined portion facing the opposite axial direction.

20. A bone fixing device as claimed in claim 13, wherein the steeply inclined portion is substantially perpendicular to the longitudinal axis.

21-23. (canceled)

24. A bone fixing device as claimed in claim 1, comprising a bioabsorbable material.

25-26. (canceled)

27. A method of treating a fracture resulting in at least two bone portions, the method comprising distracting the fracture to hold the bone portions apart under tension.

28. A method of treating a fracture as claimed in claim 27, comprising engaging a bone fixing device as claimed in claim 1 into a fractured bone to distract the fracture.

29. (canceled)

30. A method as claimed in claim 28, including the steps of inserting a guide wire into the bone and subsequently inserting the bone fixing device over the guide wire.

31-32. (canceled)

33. A jig for positioning first and second bone fixing devices, the jig defining first and second lines of insertion for the first and second bone fixing devices respectively, wherein the lines of insertion lie in parallel planes and wherein the lines of insertion cross when viewed in a direction perpendicular to the parallel planes.

34. A jig as claimed in claim 33, wherein the jig defines more than two lines of insertion.

35-36. (canceled)

37. A jig as claimed in claim 33, wherein the parallel planes have a minimum separation equal to the diameter of the bone fixing devices.

38. An assembly comprising a jig as claimed in claim 33 and first and second bone fixing devices, wherein the bone fixing devices are at least partially threaded.

39. An assembly as claimed in claim 38, wherein the parallel planes have a minimum separation equal to the outer thread diameter of the bone fixing devices.

40. A jig as claimed in claim 33, wherein the orientation of the second line of insertion is variable relative to the orientation of the first line of insertion.

41. (canceled)

42. A jig as claimed in claim 33, wherein the jig includes an arm that extends in a lateral direction with respect to at least one of the lines of insertion.

43-44. (canceled)

45. A jig as claimed in claim 42, wherein the first and second lines of insertion are defined by respective first and second guide means coupled to the arm.

46-47. (canceled)

48. A jig as claimed in claim 42, wherein the jig includes a spacer adapted to space the parallel planes of the lines of insertion apart from each other.

49. A jig as claimed in claim 48, wherein the spacer is an individual component distinct from the arm.

50. A jig as claimed in claim 48, wherein the spacer comprises a block having a guide bore adapted to receive a guide sleeve.

51. (canceled)

52. A jig as claimed in claim 48, wherein the spacer is rotatably and translatably mounted on the arm.

53. A jig as claimed in claim 42, wherein the arm is arcuate.

54. A jig as claimed in claim 48, wherein an elongate aperture is provided in the arm and the spacer is translatable along the elongate aperture.

55. A jig as claimed in claim 54, wherein the elongate aperture is arcuate, and wherein the spacer is rotationally coupled to the elongate aperture, such that as the spacer translates along the aperture, the spacer rotates with the curvature of the aperture.

56. A jig as claimed in claim 55, wherein the spacer comprises a block having a guide bore adapted to receive a guide sleeve and wherein on translation of the spacer, the spacer rotates such that the guide bore is always perpendicular to a tangent to the elongate aperture at the location of the spacer.

57. (canceled)

58. A jig as claimed in claim 42, wherein the arm comprises a first portion having a first planar surface and a second portion having a second planar surface aligned parallel to the first planar surface, and wherein the second portion is stepped relative to the first portion.

59. A jig as claimed in claim 48, wherein the spacer comprises a part of the arm.

60. (canceled)

61. A jig as claimed in claim 42, being provided with at least one guide means that is rotatably and translatably mounted on the arm.

62. A jig as claimed in claim 42, wherein the arm is extendible.

63. A method of positioning first and second bone fixing devices relative to each other, the method including the steps of defining first and second lines of insertion for the first and second bone fixing devices respectively, wherein a jig is used to define the position of the second line of insertion relative to the first line of insertion such that the lines of insertion lie in parallel planes and such that the lines of insertion cross when viewed in a direction perpendicular to the parallel planes.

64. (canceled)

65. The method as claimed in claim 63, wherein the separation of the parallel planes is at least as large as the diameter of the bone fixing devices.

66. (canceled

67. The method as claimed in claim 63, wherein the bone fixing devices are inserted at locations in which they are in contact with each other.

68. The method as claimed in claim 63, wherein the fixing devices are at least partially threaded and the separation of the parallel planes is at least as large as the outer thread diameter of the bone fixing devices.

69. The method as claimed in claim 68, wherein the separation of the parallel planes is equal to the outer thread diameter, so that when the first and second fixing devices are inserted along their respective lines of insertion, the threads on the first bone fixing device are in contact with the threads on the second bone fixing device.

70. The method as claimed in claim 63, including the steps of inserting first and second guide wires into the bone along the respective first and second lines of insertion and inserting the first and second bone fixing devices over the first and second guide wires into the bone.

71. The method as claimed in claim 63, wherein the jig includes first and second guide means which define the respective first and second lines of insertion and the method includes the step of varying the relative orientation and/or spacing of one of the guide means relative to the other.

72. (canceled)

73. A method of positioning first and second bone fixing devices in a bone such that they are in direct contact with one other.

74. A bone fixing device for treating a fracture being at least partially threaded, wherein at least one of the threads has a portion that is steeply inclined relative to the longitudinal axis of the bone fixing device, wherein the steeply inclined portion faces the leading end of the bone fixing device.

75. A bone fixing device as claimed in claim 74, wherein at least one further thread has a portion steeply inclined relative to the longitudinal axis of the bone fixing device, wherein the steeply inclined portion faces the trailing end of the bone fixing device.

76. A bone fixing device as claimed in claim 74, wherein the at least one steeply inclined portion is perpendicular to the longitudinal axis of the bone fixing device.

77. A method of treating a fracture, comprising engaging a fixing device into at least two bone portions of the fracture to maintain the relative position of the bone portions.
